(19) United States
(12) Patent Application Publication
(10) Pub. No.: US 2006/0224243 A1
(43) Pub. Date: Oct. 5, 2006

(54) Elbow prosthesis

(75) Inventors: Philippe E. Pare, Cordova, TN (US); Oludele O. Popoola, Granger, IN (US)

Correspondence Address: ZIMMER TECHNOLOGY - REEVES, P. O. BOX 1268, ALEDO, TX, 76008, US

(73) Assignee: Zimmer Technology, Inc.

(21) Appl. No.: 11/095,195

(22) Filed: Mar. 31, 2005

(51) Int. Cl. A61F 2/38

(52) U.S. Cl. 623/20.11

(57) ABSTRACT

An elbow prosthesis for replacing a portion of an elbow joint between the humerus and ulna bones.

### FIELD OF THE INVENTION

The invention relates to orthopaedic implants. In particular, this invention relates to elbow joint prostheses.

### BACKGROUND

Elbow arthroplasty has been used to restore function to diseased or injured elbow joints. The elbow joint is the articulation between the humerus or the upper arm and the ulna and radius of the forearm. The basic anatomic position of the human body is standing upright, arms hanging at the sides with palms forward. The elbow is in extension in this position. Elbow flexion occurs as the humerus is held stationary and the palm is moved upwardly pivoting the forearm about the elbow joint. Varus and valgus loading of the elbow occurs as the elbow is torqued in the coronal plane; i.e. the forearm is abducted/adducted while the humerus remains stationary. Rotating the forearm so that the radius rotates over the ulna to turn the palm toward the posterior side of the body is called pronation. Rotating the hand back to the anatomic position is called supination. Positions and directions relative to the elbow joint may be described in terms of proximal being nearer the elbow joint, distal being further from the elbow joint, anterior being nearer the front of the body on the inside of the elbow, posterior being nearer the back of the body on the outside of the elbow, medial being nearer the centerline of the body, and lateral being further from the center line of the body.

In elbow arthroplasty, a portion of the articulating surfaces of the humerus and ulna are cut away and replaced with substitute implants. In a typical case, the implants include a stemmed humeral component and a stemmed ulnar component pinned together to form a hinge joint. The bones are prepared by creating an opening down along the intramedullary canal into each bone proximal to distal. The implants may be placed directly in contact with the prepared bone surfaces for bony fixation of the implant. Alternatively, bone cement may be introduced into the prepared bones so that it hardens around and locks the components in place.

### SUMMARY

The present invention provides an elbow prosthesis for replacing a portion of an elbow joint between the humerus and ulna bones.

In one aspect of the invention, the elbow prosthesis includes a humeral component having a stem sized to fit within the intramedullary canal of the humerus and a first articulating portion. The elbow prosthesis further includes an ulnar component having a stem sized to fit within the intramedullary canal of the ulna and a second articulating portion. The first and second articulating portions form a ball and socket joint at the elbow.

In another aspect of the invention, an elbow prosthesis includes a humeral stem, a humeral articulating component, an ulnar stem and an ulnar articulating component. The humeral stem has an insertion portion sized to fit within the intramedullary canal of the humerus and a mounting portion. The humeral articulating component includes a mounting portion mountable to the humeral stem mounting portion and a first articulating portion. The ulnar stem has an insertion portion sized to fit within the intramedullary canal of the ulna and a mounting portion. The ulnar articulating component has a mounting portion mountable to the ulnar stem mounting portion and a second articulating portion. The first and second articulating portions are engageable in joint articulating arrangement.

In another aspect of the invention, a method of surgically repairing a portion of an elbow joint between the humerus and ulna bones includes: selecting a humeral stem having an insertion portion sized to fit within the intramedullary canal of the humerus and a mounting portion; selecting a humeral articulating component having a second mounting portion mountable to the humeral stem mounting portion and a first articulating portion; intraoperatively mounting the humeral articulating component to the humeral stem; selecting an ulnar stem having an insertion portion sized to fit within the intramedullary canal of the humerus and a mounting portion; selecting an ulnar articulating component having a mounting portion mountable to the ulnar stem mounting portion and a second articulating portion; intraoperatively mounting the ulnar articulating component to the ulnar stem; and engaging the first and second articulating portions in joint articulating arrangement.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is a an exploded perspective view of an illustrative elbow prosthesis according to the present invention;

FIG. 2 is cross sectional view of the prosthesis of FIG. 1 implanted in an elbow and viewed from the medial side with the elbow flexed; and

FIG. 3 is a cross sectional view of the prosthesis of FIG. 1 implanted in an elbow and viewed from the anterior side with the elbow extended.

### DESCRIPTION OF THE ILLUSTRATIVE EXAMPLES

Examples of the present invention include elbow prostheses for surgical repair of the elbow joint. The prosthesis may include a humeral component having a stem and a first articulating end and an ulnar component having a stem and a second articulating end. The first and second articulating ends may form a ball and socket joint. For example, the humeral component may include a first articulating end in the form of a ball head and the ulnar component may include a second articulating end in the form of a socket for receiving the ball head. Alternatively, the humeral component may include a first articulating end in the form of a socket and the ulnar component may include a second articulating end in the form of a ball head receivable by the socket.

The humeral and/or ulnar component may include separate modular anchorage components and articulation components that may be joined together at the time of surgery. The modular components may be joined outside of the patient's body and subsequently implanted as an assembly. Alternatively the components may be individually implanted and subsequently joined. The components may permit a variety of different articulation and anchorage components to be assembled as needed to fit a particular patients needs. For example, variations in stem lengths, diameters, materials, surface finish, and/or other parameters may be provided. Likewise, variations in ball head and socket sizes, constraint, materials, eccentric stem connection, and/or other parameters may be provided. The components may be sized and shaped to facilitate a minimally invasive surgical technique in which the individual components are inserted through a small incision and subsequently assembled. An articulation component in the form of a socket may include a separate modular socket liner.

The elbow components may be made of biocompatible materials including metals, ceramics, polymers, and/or other suitable biocompatible materials and alloys and combinations thereof. The stems can be smooth or textured and can include coatings including layers of porous materials, fiber pads, beads, plasma sprayed materials, and/or other suitable coatings. The coatings may include metals, ceramics, polymers, and/or other suitable coating materials.

Modular stems and articulating components may be joined by locking tapers, screws, bolts, adhesives, snap rings, and/or other suitable joining mechanisms. Modular components may include an indexing mechanism for aligning the components in a predetermined relative orientation. The indexing features may include projections and corresponding depressions. In particular, the indexing features may include pins, pegs, bosses, rails, undulations, holes, grooves, and/or other suitable features and combinations thereof. For example, one component may include one or more pegs projecting outwardly and another component may include one or more corresponding depressions for receiving the pegs to orient the components in a desired orientation.

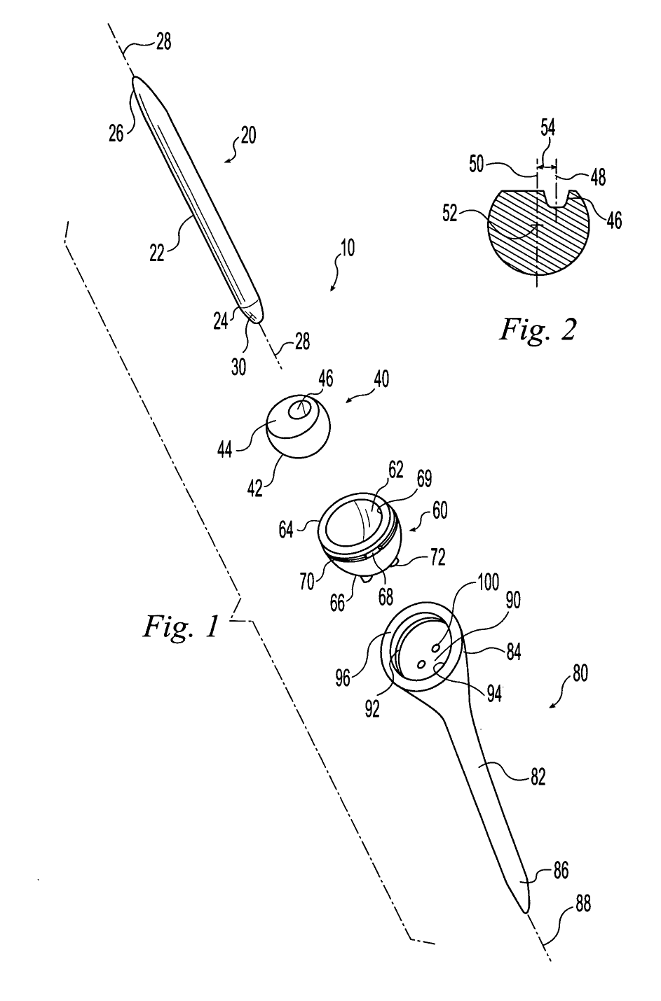

FIGS. 1-4 depict an illustrative elbow prosthesis 10 according to the present invention. The prosthesis 10 includes a humeral stem 20, a humeral articular component 40, an ulnar articular component 60, and an ulnar stem 80. The humeral stem 20 and articular component 40 may be combined in a single piece unitary construction or they may be separate modular components (as shown). Likewise, the ulnar stem 80 and articular component 60 may be combined in a single piece unitary construction or they may be separate modular components (as shown). Either of the humeral and ulnar articular components 40, 60 may form a ball head articulating surface with the other of the humeral or ulnar articular components 40, 60 forming a socket for receiving the ball head. In the illustrative example, the humeral articular component 40 defines a hemispherical ball head 42 and the ulnar articular component 60 defines a corresponding hemispherical socket 62.

The humeral stem 20 includes an elongated body 22 forming an insertion portion having a first end 24, a second end 26, and a longitudinal axis 28 extending therebetween. The first end includes an articular component 40 engaging male taper 30. Preferably, the taper 30 is of a self-locking Morse taper configuration. The second end 26 may be tapered as shown to ease insertion into the intramedullary canal of the humerus. The humeral stem 20 is provided in a variety of lengths and diameters to permit the surgeon to select the stem size that best fits a particular patient's anatomy and a variety of surface finishes and materials to permit the surgeon to select a desired method of stem fixation. The taper 30 is constant across all stem sizes to permit stems to be interchangeably connected to the ball head 42.

The ball head 42 is in the form of a hemispherical solid having a planar surface 44 formed on it. A female taper 46 is formed into the ball head 42 through the planar surface 44. Preferably the female taper 46 is of a self-locking Morse taper configuration corresponding to the male taper 30 of the humeral stem 20. The female taper 46 includes a longitudinal axis 48 and the ball head 42 includes a centerline 50 extending through the geometric head center 52 and parallel to the taper axis 48 (as shown in FIG. 2). The taper 46 may be offset 54 from the head center 52 so that the humeral stem 20 connects to the ball in an eccentric, or offset, orientation to better fit the humeral anatomy. The ball head 42 is provided in a variety of configurations of taper 46 offset 54 to allow the surgeon to select the degree of offset that best matches a particular patient's anatomy. The amount of offset 54 may vary from zero, in which case the humeral stem 20 is aligned with the head center 52, to several millimeters. The ball head 42 is also provided in a variety of materials to allow the surgeon to select a desired material. The preferred Morse taper engaging arrangement provides a single attachment configuration that can be used to join any combination of the stems 20 and heads 42.

The ulnar articular component 60 includes a body 64 defining a hemispherical socket 62 for receiving the ball head 42. The body 64 has a hemispherical back surface 66 for engaging the ulnar stem component 80. An annular groove 68 is formed in the back surface 66 near the opening 69 of the socket 62. A locking ring 70 is disposed in the groove 68 with a portion that projects outwardly from the groove around its perimeter when the ring 70 is unconstrained. The locking ring 70 is split and resilient so that it is collapsible upon engagement with the ulnar stem 80 such that it projects from the groove 68 a lesser amount. Preferably, the locking ring 70 collapses so that it is fully contained within the groove. The body 64 further includes pegs 72 projecting from the back surface 66 engageable with the ulnar stem 80 to orient the body 64 relative to the stem 80 and prevent rotation of the body. 64 relative to the stem 80. The ulnar articular component 60 is provided in a variety of materials to allow a surgeon to select a desired material. The ulnar articular component 60 may also be provided in a variety of socket 62 configurations to provide differing levels of constraint. For example the socket 62 may extend over the ball head 42 less than half the ball head radius so that is loosely connected to the ball head 42 or it may extend over the ball head 42 more than the ball head radius so that it is a snap fit onto the ball head 42.

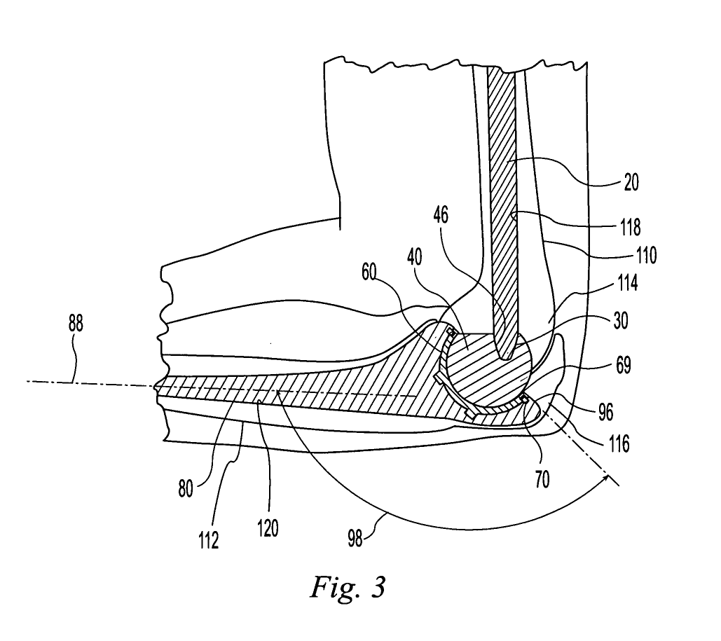

The ulnar stem 80 includes an elongated body 82 forming an insertion portion having a first end 84, a second end 86, and a longitudinal axis 88 extending therebetween. The first end 84 includes an enlarged ulnar articular component 60 engaging hollow 90. The hollow 90 is hemispherical to engage the back side 66 of the articular component 60 and includes an annular groove 92 corresponding to the groove 68 in the articular component 60. As the articular component 60 is pressed into the hollow 90, the locking ring 70 is collapsed into the groove 68 in the articular component 60. As the groove 68 in the articular component 60 and the groove 92 in the ulnar stem 80 align, the locking ring 70 expands back to its original size with a portion projecting from the articular component 60 groove 92 into the stem 80 groove 92 to lock the articular component 60 in the hollow 90. When the articular component 60 is seated in the hollow 90, the articular component opening 69 forms a socket angle 98 (FIG. 3) with the ulnar stem axis 88. Preferably, the hollow 90 opening 94 is bounded by a face 96 disposed at this same angle 98. The hollow 90 may include depressions 100 to receive the pegs 72 of the articular component 60 to orient the articular component 60 relative to the stem 80 and prevent rotation of the articular component 60 relative to the stem 80. The locking ring 70 also acts to orient the articular component 60 relative to the stem 80. The ulnar stem 80 is provided in a variety of hollow 90 configurations to provide a variety of socket angles 98 to permit a surgeon to select the socket angle that best fits a particular patient's anatomy. Preferably, the socket angle 98 is oblique, in other words neither parallel nor perpendicular, relative to the ulnar stem axis 88. The socket angle 98 causes the socket 62 to face the humerus in a more anatomical orientation. The second end 86 of the ulnar stem 80 may be tapered as shown to ease insertion into the intramedullary canal of the ulna. The ulnar stem 80 is provided in a variety of lengths, diameters, and socket angles to permit the surgeon to select the stem that best fits a particular patient's anatomy and a variety of surface finishes and materials to permit the surgeon to select a desired method of stem fixation. The hollow 90 is constant across all stem sizes to permit stems to be interchangeably connected to the ulnar articular components 60.

The ball and socket configuration of the illustrative example permits three degrees of rotational freedom which facilitates increased varus/valgus and pronation/supination ranges of motion of the elbow compared to prior art hinge-type elbow prostheses which permit only a single degree of freedom. In addition, the relatively large contact area of the articular components 40, 60 of the present invention result in relatively lower contact stresses between the components 40, 60 to improve the longevity of the prosthesis. The modular construction permits different combinations of components to provide for a desired fit to a patient's anatomy, desired materials, and desired level of constraint.

In use, the illustrative elbow prosthesis 10 is implanted by first preparing the humerus 110 and ulna 112 (FIG. 3) by removing a proximal portion 114, 116 of each bone and exposing the intramedullary canal 118, 120 of each bone. A humeral stem 20 is selected that has an insertion portion sized to fit within the intramedullary canal 118 of the humerus 110. In addition to size, the stem 20 may be selected based on material, surface finish, and/or other selection criteria. A humeral articulating component 40 is selected based on material choice, size, constraint, and/or other selection criteria. The humeral articulating component 40 is intraoperatively mounted to the stem 20 by engaging the stem taper 30 with the articulating component 40 taper 46. The components are impacted together to lock them together. An ulnar stem 80 is selected that has an insertion portion sized to fit within the intramedullary canal 120 of the ulna 112. In addition to size, the stem 80 may be selected based on material, surface finish, and/or other selection criteria. An ulnar articulating component 60 is selected based on material choice, size, constraint, and/or other selection criteria. The ulnar articulating component 60 is intraoperatively mounted to the stem 80 by inserting the ulnar articulating component 60 into the hollow 90 in the stem 80 and aligning the pegs 72 with the corresponding depressions 100. As the ulnar articulating component 60 is pressed into the hollow 90, the locking ring 70 is collapsed. When the groove 68 in the ulnar articulating component 60 aligns with the groove 92 in the hollow 90, the locking ring 70 expands and locks the components together. The stems 20, 80 may be inserted into the prepared bones 110, 112 before or after the articulating components 40,60 are mounted to the stems 20, 80. Once the components are assembled and inserted, the prosthesis forms a ball and socket joint articulation at the elbow between the articulating components 40, 60.

Although an illustrative elbow implant and its use has been described and illustrated in detail, it is to be understood that the same is intended by way of illustration and example only and is not to be taken by way of limitation. Accordingly, variations in and modifications to the implants and their use will be apparent to those of ordinary skill in the art, and the following claims are intended to cover all such modifications and equivalents.

What is claimed is:

1. An elbow prosthesis for replacing a portion of an elbow joint between the humerus and ulna bones, the bones having intramedullary canals, the prosthesis comprising:
a humeral component having a stem sized to fit within the intramedullary canal of the humerus and a first articulating portion; and
an ulnar component having a stem sized to fit within the intramedullary canal of the ulna and a second articulating portion, the first and second articulating portions forming a ball and socket joint at the elbow.

2. The elbow prosthesis of claim 1 wherein the first articulating portion comprises a hemispherical ball head and the second articulating portion comprises a hemispherical socket engageable with the ball head.

3. The elbow prosthesis of claim 1 wherein the first articulating portion comprises a hemispherical socket and the second articulating portion comprises a hemispherical ball head engageable with the socket.

4. The elbow prosthesis of claim 1 wherein the first articulating portion comprises a separate modular component mounted to the humeral stem.

5. The elbow prosthesis of claim 4 wherein the first articulating portion comprises a hemispherical ball head.

6. The elbow prosthesis of claim 5 wherein the ball head includes a geometric head center and the ball head is mounted to the humeral stem in an offset position in which the stem is not aligned with the head center.

7. The elbow prosthesis of claim 6 wherein the ball head is one of a plurality of ball heads provided in a set of ball heads interchangeably mounted to the humeral stem, each of the plurality being mountable to the humeral stem in a different offset position.

8. The elbow prosthesis of claim 6 wherein the humeral stem includes a male taper and the ball head defines a female taper engageable with the male taper in self-locking configuration.

9. The elbow prosthesis of claim 1 wherein second articulating portion comprises a separate modular component mounted to the ulnar stem.

10. The elbow prosthesis of claim 9 wherein the second articulating portion comprises a body defining a hemispherical socket.

11. The elbow prosthesis of claim 10 wherein the socket includes an opening and the ulnar stem includes a longitudinal axis, the socket opening being oblique to the stem axis.

12. The elbow prosthesis of claim 11 wherein the hollow comprises an indexing mechanism for aligning the socket and hollow in a predetermined relative orientation.

13. The elbow prosthesis of claim 12 wherein the stem includes hollow and a first annular groove, the body further including a backside surface defining a second annular groove, the backside surface being engageable with the hollow with the first and second annular grooves being aligned, a locking ring being disposed in the annular grooves to lock the body and stem in engagement.

14. The elbow prosthesis of claim 13 wherein the body comprises at least one outwardly projecting peg and the hollow includes at least one corresponding depression, the peg being engageable with the depression to orient the body relative to the stem.

15. An elbow prosthesis for replacing a portion of an elbow joint between the humerus and ulna bones, the bones having intramedullary canals, the prosthesis comprising:
a humeral stem having an insertion portion sized to fit within the intramedullary canal of the humerus and a mounting portion;
a humeral articulating component having a mounting portion mountable to the humeral stem mounting portion and a first articulating portion;
an ulnar stem having an insertion portion sized to fit within the intramedullary canal of the ulna and a mounting portion; and
an ulnar articulating component having a mounting portion mountable to the ulnar stem mounting portion and a second articulating portion, the first and second articulating portions being engageable in joint articulating arrangement.

16. The elbow prosthesis of claim 15 wherein the first and second articulating portions define a ball and socket joint.

17. The elbow prosthesis of claim 15 wherein the humeral stem, humeral articulating component, ulnar stem, and ulnar articulating component are each provided in a plurality of configurations intraoperatively assemblable into a desired prosthesis configuration.

18. The elbow prosthesis of claim 15 wherein the humeral articulating component comprises a ball head mountable on the humeral stem, further wherein the ulnar stem comprises a hollow and the ulnar articulating component comprises a body defining a socket, the body being mountable within the hollow and the socket being engageable with the ball head.

19. A method of surgically repairing a portion of an elbow joint between the humerus and ulna bones, the bones having intramedullary canals, the method comprising:
selecting a humeral stem having an insertion portion sized to fit within the intramedullary canal of the humerus and a mounting portion;
selecting a humeral articulating component having a second mounting portion mountable to the humeral stem mounting portion and a first articulating portion;
intraoperatively mounting the humeral articulating component to the humeral stem;
selecting an ulnar stem having an insertion portion sized to fit within the intramedullary canal of the ulna and a mounting portion;
selecting an ulnar articulating component having a mounting portion mountable to the ulnar stem mounting portion and a second articulating portion;
intraoperatively mounting the ulnar articulating component to the ulnar stem; and
engaging the first and second articulating portions in joint articulating arrangement.
